(19) United States
(12) Patent Application Publication
(10) Pub. No.: US 2016/0257815 A1
(43) Pub. Date: Sep. 8, 2016

(54) METHOD OF MAKING LIGNOCELLULOSIC COMPOSITES

(71) Applicant: Solenis Technologies, L.P., Schaffhausen (CH)

(72) Inventor: Daniel F. Varnell, Wilmington, DE (US)

(21) Appl. No.: 15/059,568

(22) Filed: Mar. 3, 2016

Related U.S. Application Data
(60) Provisional application No. 62/127,867, filed on Mar. 4, 2015.

(51) Int. Cl. C08L 97/02; C12N 9/80

(52) U.S. Cl. CPC C08L 97/02; C12N 9/80; C12Y 305/01005

(57) ABSTRACT

A method of preparing a lignocellulosic composite comprising combining one or more lignocellulosic materials with a liquid curative, followed by the addition of one or more dry protein sources, forming the composition into a composite structure and then curing the composite structure.

This application claims the benefit of US provisional application No. 62/127867, filed 4 Mar. 2015, the entire contents of which are hereby incorporated by reference.

### BACKGROUND OF THE INVENTION

The current invention is directed to processes of preparing lignocellulosic based composites, which are bonded with an adhesive, wherein a dry or powdered protein source is added to a mixture of a lignocellulosic material and curing agent wherein the curing agent is in liquid form when added to and mixed with the lignocellulosic component.

In most composite manufacturing processes that utilize an adhesive, the adhesive portion will set-up or go from being in a liquid state to a solid state. The adhesive may set-up by: loss of water into the air or into another portion of the composite; by a phase change; or by some chemical or physio-chemical change of the adhesive.

Many adhesives in the composite industry, especially where biomaterials are used, are water-borne. In this situation, water serves as a primary component either to dissolve or disperse the adhesive components. For example urea-formaldehyde (UF) adhesives are often provided in the form of a solution or dispersion in water. Most latex adhesives are water-based emulsions or dispersions and most protein based adhesives used in the composite industry are water based. Many of the water based adhesives such as soy flour based adhesives are described in numerous books, articles, and patents, for example, U.S. Pat. No. 7,060,798 and U.S. Pat. No. 7,252,735. Similar work has been done using urea formaldehyde (UF) based adhesives, for example, melamine urea formaldehyde (MUF) adhesives, melamine formaldehyde (MF) adhesives, phenol formaldehyde (PF) adhesives, and poly(vinyl acetate) and poly(ethylene vinyl acetate) adhesives.

Many of the formaldehyde based resins, such as urea formaldehyde resins, are strong, fast curing, and reasonably easy to use. However, such resins lack the ability to resist chemical decomposition in the presence of water or lack hydrolytic stability along the polymer backbone. Some of the issues associated with these types of resins are that there can be significant amounts of free formaldehyde released from the finished products (and ultimately the formaldehyde can be inhaled by the occupants within a home). Because of this, there have been several legislative actions to push for the removal of these resins from interior home applications (Health and Safety Code Title 17 California Code of Regulations Sec. 93120-93120.12). The fact that the present processes uses protein based adhesives and do not require formaldehyde is a significant advantage over formaldehyde based resins.

In many of the current processes being used in composite manufacturing today, protein sources, such as soy protein isolate or soy flour, are used in combination with a curative. The curative may also be called a crosslinking agent or catalyst. The curative reacts with or it reacts in the presence of the protein source to achieve superior properties of the composites, such as improved bending strength or water resistance versus use of the protein alone. Soy protein based adhesives are described in U.S. Pat. No. 7,736,559, U.S. Pat. No. 7,345,136, U.S. Pat. No. 7,393,930, U.S. Pat. No. 7,252,735, U.S. Pat. No. 7,060,798, U.S. Pat. No. 6,497,760, U.S. Pat. No. 6,306,997, U.S. Pat. No. 1,994,050, U.S. Pat. No. 1,813,387, U.S. Pat. No. 1,724,695, and US Patent Application No. 2007/0148339. Information on soy adhesives can also be found in articles such as Yang et al. Comparison ofprotein-based adhesive resins for wood composites, J Wood Sci. (2006) 52: 503-508 and Kumar et al. Adhesives and plastics based on soy protein products, Industrial Crops and Products 16 (2002) 155-172, and in published applications WO 2010/065758 A2, WO 2007/064970 A1, and WO 2008/118741 A1.

The above references describe how soy protein or soy flour is dispersed in water and then mixed with a curative, and possibly other additives, to yield adhesives that can be rolled, sprayed, or otherwise applied to the lignocellulosic components to form a composite —as for example, in preparation of particleboard, or medium density fiber (MDF) board where the adhesive is sprayed onto and/or mixed with the lignocellulosic component.

One aspect of the present process is the addition of the protein source as a powder to the lignocellulosic material wherein the addition of the powdered protein source occurs separately from the application of a curative in liquid form to the lignocellulosic material and generally added subsequent to any mixing or blending of the lignocellulosic material and curative liquid. For example, the adhesive can be added subsequent to the blender in particle board manufacturing or subsequent to the blowline in MDF manufacturing.

Generally, when a protein and curative are used in composite manufacturing, the protein source and the curative are mixed together before being applied to a lignocellulosic material or the protein source is first dispersed in water. U.S. Pat. No. 6,306,997 by Kuo described soy-bean based adhesive formulations of neutral pH. The process he described begins with making an aqueous solution of soybean flour. U.S. Pat. No. 7,060,798 by Li uses soy flour but starts with the preparation of an aqueous adhesive composition which once made is mixed with the lignocellulosic materials.

U.S. Pat. No. 6,790,271 describes an adhesive based on a mixture of soy protein isolate, a polyol plasticizer, and a vegetable oil derivative. More particularly the references relates to a water resistant soy protein based adhesive containing a vegetable oil derivative. The reference teaches that the materials, including the curative, must be premixed to produce a homogeneous powder where the particles easily separate. This blend is then mixed with wood in the preparation of particleboard. This reference teaches that the protein requires plasticizers such as polyols and vegetable oil derivatives to initiate flow.

When an adhesive is sprayed onto the lignocellulosic material of a composite there will be certain viscosity restrictions dictated by the spraying process. For example in the manufacturing of a particleboard the adhesive is sprayed onto the lignocellulosic material (typically wood) and then the mixture is further blended. In this type of process the adhesive formulation must be in a sprayable form. A sprayable form means that the viscosity must be below a certain threshold and is dependent upon the mill

With some water and protein based adhesives, including soy flour, the viscosity of the solution or dispersion can increase rapidly with the protein concentration and cause spraying and/or runnability issues. Therefore, the concentration must be kept low to allow for proper flowability. At the same time, water-based adhesives also have lower limitations on the concentration of solids that can be used. Too much water added to the composite by the adhesive can prevent the successful manufacturing of the composite. For example, there could be too much shrinkage, or if curing by hot-pressing, too much steam pressure may build inside the formed structure and lead to delaminating of the structure or blowing apart of the structure when pressure is released. Both are common problems in the manufacturing of particleboard. The current process resolves these and other issues, such as high viscosity, associated with making a dispersion or solution of adhesive that contains a protein source, such as soy flour, by the separate addition of the protein source in powder form.

As seen above, traditional composite board manufacture dictates the addition of a protein source in liquid form in combination with a curative. In traditional processes the mixing of the components will begin the curing process and thus limit the pot life (or working time) of the adhesive after it has been prepared for application to the lignocellulosic material. Separate addition of the protein source and the curative eliminates this issue.

Matthew John Schwarzkopf, Kaichang Li at Oregon State University, in Apr. 8, 2009, published a thesis which describes a “dry method” for composite manufacturing where soy flour is added to a wood furnish prior to application of a curative. In Schwarzkopf's process the mixing of the soy flour (a protein source) must be prior to the addition of a curative. This same process is later found in a paper by Li, “Effects of Adhesive Application Methods on Performance of a Soy-based Adhesive in Oriented Strandboard” J. Am. Oil Chemists Soc., October 2009, 86{10), pp. 1001-1007. Li teaches the soy flour and wood furnish are pre-mixed prior to application of the curative and specifically that the “dry method” involved spraying the CA (curing agent) solution onto a mixture of SF {soy flour) and wood flakes.”

However, it is difficult to move and meter the protein source in dry form to a place where it can be added to the lignocellulosic material after the lignocellulosic material has passed over a weighing belt and before it enters a process where a liquid adhesive is applied, or in the case of the “dry-method”, where a liquid curative is applied. Furthermore, addition of the dry protein source into the same process step where the liquid curative is applied, such as in a blender, can lead to contact of the protein source powder with the liquid, which can lead to deposits in the process equipment, such as in a blender. It is also difficult to add the dry powder protein source into a stage such as a blow-line where lignocellulosic material is sprayed with liquid adhesive as in the case of preparation of medium density fiberboard.

While traditional methods of composite board manufacture dictate adding the protein source to the lignocellulosic material prior to adding a curative, we have discovered that adding a dry or powdered protein source subsequent to and separately from the mixing of the lignocellulosic and curative has unexpected benefits.

U.S. Pat. No. 7,736,559 (the '559 patent) describes compositions of adhesives based on a protein source and a polyamidoamine/epichlorohydrin curative. Moisture limits, well known to the industry for the manufacturing of composites, are stated as being a limitation in the manufacturing of wood composites. Included are the typical wood moisture, solids contents of a curative, and typical adhesive addition levels. The reference teaches a mixture of wood, amine curative, and a protein source having a total moisture input, defined by a particular equation, of between 8 and 12% of the wood component. The ‘559 patent discloses that to obtain this level of moisture the protein source, can be added as a dry powder to an aqueous curative. However, addition of a powder protein component into a blender where liquid adhesive is being added can lead to deposits. Whereas, the process of the current method separates the addition points of the liquid curative and the protein source thus alleviating these issues.

The use of diluents or plasticizers with proteins in the manufacture of composites is well documented. The high viscosity of protein materials and the need to limit water has been addressed by the addition of diluents, as described in US Patent Publication 2009/0098387. Water is replaced by diluents in a solution or dispersion of protein thus keeping both the protein level and the water level low. A combination of a certain level of diluents and a limit to the soy flour level are needed to keep the viscosity low and the moisture level low.

The addition of urea as an additive is also known. U.S. Patent Publication 2010/0069534 A1, by Wescott, describes the advantages of adding urea to a soy flour based composite adhesive. The urea is added to a wet composition of soy flour. It can be a diluent, but it may be more accurately described as an aid to denature the soy protein and thus enhance properties. Although soy protein isolates, generally referred to as soy protein, do not contain urease, soy flour can be processed at a low temperature, containing the enzyme urease which would break down the urea into ammonia and carbon dioxide. Urease may be eliminated with heat treatment. U.S. Patent Publication No. 2011/0048280, utilizes an acidification to eliminate urease enzyme. The process needed to kill or inhibit the urease adds complexity and cost to the process of using soy flour or other urease containing protein sources. U.S. Pat. No. 5,582,682, discloses a process for making cellulosic composites with a thermoset adhesive that utilizes the Maillard reaction where both the carbohydrate for the Maillard reaction and a solids-residue are derived from the same particulated cellulosic feedstock. In the process the cellulosic feedstock and a protein containing material are mixed and treated with ammonia to obtain the composite forming mixture. The ammonia must be present in high enough concentration to increase the pH to alkaline and start the Maillard reaction which is seen to occur by a deep darkening of the materials. The process is considered dry because there is no free liquid. The “no liquid” adhesive of the patent is premade and then applied to the solids residue. In the current process the cellulosic and the protein source are not from the same feed stock and the total adhesive is not premade.

WO 2010/065758 discloses soy protein composites with a particular type of curing agent. The patent application teaches that a soy protein, curing agent, and comminuted lignocellulosic material may be mixed in any order and that the curing agent and lignocellulosic can be premixed prior to mixing with the soy protein. However, in this process the materials are dispersed or dissolved. Reference WO 2007/064970 discloses blending glycerol with soy protein isolate producing a homogenous powder. However, the dry adhesive material is an adhesive formulation containing both protein and curative. Furthermore, the protein must be denatured prior to use.

In one aspect of the current invention urea is present in the adhesive and urease is present in the protein source. Although urease is generally found in the protein source, urease is not always present, for example in soy protein isolate, normally referred to as soy protein. In WO 2008/118741, urea is used as a control for splice-line staining in a wood composite and in the application both soy protein and a curing agent are present in the adhesive. Urea is not the only material that can be used in the application, but when urea was used it was noted that it gave a pH increase that could be adjusted by addition of an appropriate acid or buffering agent. The levels of urea used in the application were quite low, with the highest level being 2% of the soy flour and thus a very low level of urea is present in the composite composition. This is very different from the use of urea at a high level as a diluent, as in the case of the current invention. If a high level of urea had been used, as was used in WO 2008/118741, then the generation of ammonia would have been overwhelming. The process of the current invention allows for addition of a high level of urea.

In one aspect of the present process, the level of urea on a dry basis is from about 1 part to about 4 parts per 100 parts lignocellulosic material.

Most composites are shaped prior to the setting-up of the adhesive. In operations, such as the manufacturing of particleboard, layers of treated biomaterial, such as wood particles or chips, are formed, then cold pressed to form an uncured mat, and then hot-pressed to set-up the adhesive. For such cases the molded form is a large planer sheet. For other applications the shape might be more complex, such as that of a flower pot or door molding.

In some processes, the shaped part must have some integrity even before the adhesive has set-up. This structural integrity may be referred to as green strength or cohesive strength or tack. Tack is the term typically used at a particleboard manufacturing site. Tack can also refer to the impartation of such cohesive strength by the adhesive portion of the composite. In the formation of something like a composite structure in the shape of a bowl the need for tack would be required if the bowl were free standing during the process when the adhesive has yet to set. Tack is also required even of materials formed into sheets. For example, on some particleboard manufacturing lines the formed mat is divided into sheets of the size of a final board product in the planer directions and then the sheets travel to a heated press. As the boards travel to the press they may span a gap and be unsupported for a time (a line without a caul). In this and other operations the uncured shaped composite structure requires some cohesive strength for the shape of the uncured composite to be maintained and to be void of cracks or fissures or other defects that might occur because of a lack of tack. Often an adhesive, for the composites, is said to have bad or good tack properties. For many applications the lignocellulosic provides no tack and does not retain a structure as an uncured material in the absence of an adhesive.

It is recognized in the composite industry that some adhesives provide good tack and others do not. The effect of the composition of UF (urea formaldehyde) adhesives on tack is discussed in an article by Leichti, Hse, and Tang, “J. Adh, 1988, pp 31-44. UF resins are generally considered to have good tack. MDI based adhesives are generally considered to have very poor tack. Some environmentally friendly adhesives can have good tack and tack often depends on the protein level. However, if the protein is first dissolved or dispersed in water, the level at which the protein source is added to the adhesive is very limited before viscosity becomes unmanageable, especially for adhesives that are sprayed on the lignocellulosic material. Therefore, there is a need for a protein based adhesive with a high enough level of protein to lead to good tack of the treated lignocellulosic material.

From all the above it is apparent that issues remain with the processes used to make composite panels, including cases where a protein source is used with a curative in liquid form and lignocellulosic material. The current process addresses these and other issues.

However, there still exists a need to be able to manufacture composites and avoiding issues such as, being able to accurately weigh the amount of protein source to be added when the weighing of the wood is immediately prior to the blender or blowline in a composite manufacturing process. Also avoided, is the development of deposits from the combination of free dry protein source being added in the same process step where a curative in liquid form is added, such as inside a process line blender or blow-line. It has been found that by waiting to add a dry protein source subsequent to the combining and mixing of a lignocellulosic material with a curative, a superior composite can be formed and produced without encountering the deficiencies of known processes.

### BRIEF SUMMARY OF THE INVENTION

The current method provides for a process of preparing lignocellulosic based composites, which are bonded with an adhesive comprised of a protein source and a curative. More particularly, the protein source is added as a powder to the lignocellulosic component after and separately from the application of a curative.

The present process also relates to the manufacture of a composite wherein a dry or powdered protein source is combined with a lignocellulosic material after the combining and/or mixing of any liquid curative component of the adhesive with the lignocellulosic material, and in a separate process step from the addition of any other liquid components.

Using the present method, it was found that a uniform distribution of the protein source and the lignocellulosic component is still obtained despite the pre-mixing and separate treatment of the lignocellulosic material with the curative in dispersed or liquid form. The fact that uniform distribution of the powder protein source throughout the composite when the protein is added after and separately from the blending steps is completely unexpected.

The current process is also directed toward the production of composites where the adhesive is free of formaldehyde in the final composition and in the chemical production of the adhesive. It is also directed toward the scavenging of the formaldehyde generated from the lignocellulosic material of the composite.

Additionally, the present process provides a means in the manufacturing of lignocellulosic composites for reducing or eliminating issues with the dispersing of a protein source in water and the limitations of solids content of a protein and curative dispersion. The current process also provides for reducing or eliminating problems with trying to spray a high viscosity solution or dispersion of a protein source. The invention also reduces or eliminates the need to neutralize urease in the protein source before it is used as an adhesive when urea is part of the adhesive formulation or part of the composite.

In some embodiments the current process may lead to better tack during forming of the composites.

In other embodiments of the current method, the process provides enhanced strengths of the final composites over the same formulation prepared as a water-based adhesive where a soy dispersion is prepared.

Additionally, the method provides for one to obtain low moisture content for the entire treated composite prior to pressing or curing thus eliminating issues associated with too much moisture. For example, addition of a protein based adhesive of 50% solids at 10 parts dry weight basis of the dry wood with 90 parts wood, dry basis, having 4% moisture content, would lead to a total moisture of 13.8 parts water to 100 parts dry wood. If instead 7 parts soy flour at 93% solids was added to the wood and separately 3 parts of a curative portion at 50% solids was added, then the final total moisture content is 7.3 parts water to 100 parts wood.

In some aspects of the current process the level of curative on a dry basis is from about 30 parts to about 100 parts per 100 parts protein source.

Another result of the present method is an increase in the pH of the formed composite or the treated furnish during the process of making the composite. Finally, provided is a curative that performs better as a result of increased pH, which results from the combination of urea and urease. Because the protein source is added in dry or powdered form after and separate from the addition of the curative to the lignocellulosic material, the development of ammonia is delayed and away from the point where liquid materials are applied and closer to the curing step of the overall composite allowing for better and higher curing rates and allows for a safer environment.

The current method also provides for a process wherein the pH of the mixture of lignocellulosic material and curative increases from the time the lignocellulosic is mixed with the curative and the addition of the powdered protein source to the time the furnish is pressed into the final composite. Lignocellulosic-based composites, such as particleboard, are prepared from combinations of a lignocellulosic such as wood, and a binder, also known as a resin and also known as an adhesive. Therefore, a lignocellulosic composite comprises a lignocellulosic material held together by an adhesive.

### DETAILS OF THE INVENTION

In some aspects, the current method is directed toward processes and materials used in the production of lignocellulosic composites. More particularly, it is directed to a method of preparing a lignocellulosic based composite by combining a lignocellulosic material with one or more curatives wherein at least one of the one or more curatives is dissolved or suspended in water thereby forming a mixture of lignocellulosic material and curative. To this mixture at least one powdered protein source is added prior to the composite being formed into a shape. The formed composite is then cured by typical techniques known in the industry. In other aspects, additional additives can be added to the mixture of lignocellulosic material and one or more liquid curatives.

In another aspect, the curative is added to the lignocellulosic material in the blender of a composite manufacturing facility and the powdered (dry) protein is added subsequent to the blender. In another aspect, the curative is added to the lignocellulosic material in a blow line of a fiber board line and the powdered (dry) protein is added subsequent to the blow line.

In another aspect, at the time of the addition of the protein source to the mixture of the lignocellulosic material and curative, the protein source is dry and the lignocellulosic material, although treated with curative and optionally other additives, is still present as discrete particles, chips, flakes or fibers. This means there is little or no adhesion between the particles, chips, fibers or flakes or between the individual particles of the powdered protein source or between the protein source and the lignocellulosic after application and prior to any pressing. It is believed that the protein source powder adheres lightly to the lignocellulosic material by electrostatic charges and/or the natural attraction of a dry powdery material to a damp surface.

In the current process the addition of the protein source to the mixture of lignocellulosic material and curative may be accomplished by any means known in the art. One surprising result of the present process was how easily and uniformly powders of protein sources mix with free flowing lignocellulosic fibers, particles and flakes. The dry or powdered protein source can be added to the lignocellulosic mixture at any point of the composite manufacturing process as long as it is after the liquid curative has been combined with the lignocellulosic material. The means of conveyance of the lignocellulosic material can be any means known in the art for transporting a relatively dry, free flowing volume of lignocellulosic material in the form of, for example, flakes, fibers, chips, irregular pieces, powder, mulch, and dust and the conveyance means can include conveyors, moving buckets, pneumatic lines, screw feeds, as well as other means known in the art.

Likewise, the protein source can be conveyed to the lignocellulosic material by means similar to those described above. The amount of protein source is calculated and/or metered according to the flow or transport rate of the lignocellulosic material. Again, such means are well known in the area of technology of moving and metering dry powder materials.

Any method of combining the protein source with the lignocellulosic/curative mixture can be used for bringing the two materials into contact with each other and providing mixing, such as a tumbling action or shaking, or mixing may be accomplished by turbulent mixing, or any type of mixing in between. Once the protein source is in contact with the lignocellulosic material, it has a relatively strong attraction to the lignocellulosic material, such that it is not removed in a high speed pneumatic line, or by a cyclone separator typically found at the end of a pressurized line.

In the current process the protein source is not added prior to or simultaneously with a liquid curative or any other liquid component, because contacting of the protein source as a free powder, can lead to agglomerations of the protein source. In addition, the protein source is not to be added to the lignocellulosic material prior to the flow rate of the lignocellulosic material being measured, nor it is not added prior to the lignocellulosic being treated with the curative. The measurement of the flow rate of the lignocellulosic material and its treatment with a curative often happen in close proximity. In some embodiments no other additives are combined with the lignocellulosic material between addition of the powdered protein source and the forming of the treated lignocellulosic into its final shape.

In the current process the protein source and the curative are never premixed prior to the addition of the protein source to the lignocellulosic material of the composites. It was totally unexpected that when the protein source was added to the lignocellulosic material, separately from the curative that it would lead to equal or better mechanical properties of the final adhesive. It was unexpected that a separate addition of a powdered protein source, especially after treatment of the lignocellulosic by the curative, could be a viable process for obtaining good adhesive curing, which is essential for good mechanical properties.

Another aspect of the process is that a urea would be combined with the lignocellulosic material separately or as part of the curative, and this application would occur prior to and separately from the addition of the protein source. This is especially significant when the protein source contains urease, such that a reaction occurs between urea and urease and results in the production of ammonia, which results in an increase of the pH of the adhesive. With certain curatives this can result in faster and/or more complete curing. For example, a polyamidoamine-epichlorohydrin resin (PAE resin) will react faster at a pH of 7 compared with a pH of 5.

If a protein source containing urease was prepared as a dispersion or solution in water and that same adhesive mixture contained urea, then a rapid generation of ammonia would occur as the urease acted on the urea. The result of such interaction is generation of ammonia and CO2 gas which could pressurize a sealed container. This results in significant safety issues. In addition, a rapid increase of pH would occur which could compromise the adhesive formulation and its performance. In the current process a formed composite may have ammonia generated if there is urease in the protein source and urea is on the lignocellulosic material. In the current process of a post and separate addition of urease containing protein source, the rate and level of ammonia released is reduced and there is less chance for a problematic ammonia smell.

### Composite Formulations

Composites are composed of multiple materials, typically of a primary material such as wood, and other secondary materials, such as fiber or filler forming the composite which is held together by an adhesive. An adhesive used for composites may also be referred to as a binder or resin.

In one aspect of the present invention, the lignocellulosic material, which comprises the major portion of the composite, ranges from about 40% to about 99% by volume, can be from about 55% to about 98% by volume, can be from about 70% to about 98% by volume and may be from about 80% to about 98% by volume. The adhesive portion of the composition, comprising the curative and the protein source, can be from about 1% to about 60% of the composite by volume, can be from about 2% to about 45% by volume, can be from about 2% to about 30% by volume and may be from about 2% to about 20% of the composite by volume. Other materials may be added in place of the lignocellulosic or the adhesive component, which can alter the level of lignocellulosic and/or adhesive.

In the current process a lignocellulosic material is used as the primary material. The most common lignocellulosic is wood, but other lignocellulosic natural materials can be used such as plant stalks, plant waste, bamboo, sugar cane based material, cellulose fibers such as pulp used in paper making, cellulose fibers, bagasse, kenaf, flax, ramie, hemp, sisal, abaca, palm, jute, soy bean hulls, nut shells, cotton, zein, rapeseed meal or any combinations thereof. The lignocellulosic materials can also be combined with synthetic fibers, flakes, and fillers. Modified or carbonized forms of the natural materials can also be used.

The lignocellulosic materials may come in various forms and shapes such as fibers, flakes, chips, particles, shavings, puffed material, stalks, and dust. In one embodiment of the invention, a portion or all of the lignocellulosic materials are wood based. When the lignocellulosic component of the composite is wood it can be in the form of wood fibers, dust, particles, chips, and/or flakes.

In one embodiment, the lignocellulosic material is in the form of chips, particles and/or dust. In another embodiment the formed composite is a particleboard. In another embodiment, the lignocellulosic component is wood fibers such as used in the manufacturing of medium density fiber board (MDF).

The moisture content of lignocellulosic materials in the form obtained or in natural form or in the form after processing or purifying may vary. Therefore, it is common practice to control, usually by drying, the moisture content of the lignocellulosic material. In one embodiment of the current process, drying can be done prior to addition of any curative, and thus prior to addition of the powdered protein source. In another embodiment drying occurs after addition of the curative and after a separate addition of the powdered protein source. In yet another embodiment, the drying occurs after the addition of the curative but prior to the addition of the protein source.

In some aspects, the lignocellulosic material will have a moisture content of from about 2% to about 8%, can be from about 3% to about 8%, and may be from about 3% to about 6%.

In other aspects of the current method the lignocellulosic materials are held together or bonded together or glued together by an adhesive. For many lignocellulosic composites the most common adhesives are urea-formaldehyde resins and phenol formaldehyde resins. The current method is applicable to adhesives that are based on a protein source mixed with a curing agent otherwise called a curative, curing agent or accelerator. Suitable protein sources or components on which the adhesive may be based include, but are not limited to, soy protein isolate, soy protein concentrate, soy flour, corn gluten meal, whey protein, wheat gluten, dried egg whites, gelatin, peanut flour, lupin flour, other high protein flours, feather meal, keratin, blood meal, collagen, gluten, casein, and spirulina. Various grades of these materials are included.

In other aspects, the protein source is soy flour. For example soy flours come with different Protein Dispersibility Indexes (PDI), for example 90 or 20. The PDI is a means of comparing the solubility of a protein in water, and is widely used in the soybean industry. One well known test in the industry, soy flour is mixed with water in a specific manner and the level of protein in the starting material and in the water phase is determined. For instance, a PDI of 100 indicates 100% of the protein is in the water phase. PDI is affected not only by the type of protein source used, but also by the manufacturing processes. For instance, heating can lower the PDI of a soybean sample.

There can be certain advantages to using different PDI materials. For example, the PDI of the protein source (as measured by the method used for soy flours) can be greater than 50, can be 70 or above, can be above 80 and may be 90 or above. A mixture of protein sources may be used and may provide advantages over the use of a single material. In the current method the protein source can be in powder form which is capable of being dispersed onto or integrated uniformly with the lignocellulosic component. The average particle size of the protein source can be less than 200 microns, can be less than 100 microns, can be less than 50 microns and may be less than 25 microns as measured by techniques used in the industry such as through optical analysis using for example Malvern Instruments Sysmex FPIA 3000 Flow Image Particle Analyzer. Other techniques include screening or partitioning techniques. The protein source(s) may be pretreated or modified by known methods such as those taught by Weining Huang et al., Adhesive Properties of Soy Proteins Modified by Sodium Dodecyl Sulfate and Sodium dodecylbenzene Sulfonate, Journal of Oil & Fat Industries (Impact Factor 1.62), 06/2000, 77(7):705-708; Guangyan Qi et al., Adhesion and Physicochemical Properties of Soy Protein Modified by Sodium Bisulfite, Journal of the American Oil Chemists’ Society (JAOCS), December 2013, Vol. 90, No. 12, p1917; and Xiuzhi Sun et al., JAOCS, 1999, vol. 76, No. 8, p 977; as long as the protein can be returned to a dry powder form prior to use.

The level of protein source needed in the current process depends on the percentage of protein in the source material. For example soy flour is typically about 50% protein by weight whereas a soy concentrate may contain about 70% protein. For the current process the actual protein level (not the level of protein source) added to the composite on a dry weight percent of the lignocellulosic component can be from about 0.5% to about 15% and can be from about 0.6% to about 10%; can be from about 0.8% to about 5%; and may be from about 0.8% to about 3%.

In one embodiment, when soy flour or lupin flour is used as the protein source, the level of the protein source can be from about 1.6 parts to about 10 parts per hundred parts of the lignocellulosic material, on a dry weight basis, and can be from about 1.6 parts to about 6 parts per hundred parts lignocellulosic material, on a dry weight basis. In one embodiment the protein source is a soy flour or lupin flour and the level is from about 4 parts and 6 parts per hundred parts of the lignocellulosic material, on a dry weight basis. The soy flour can be a high dispersability soy flour having a PDI of greater than 50 and the PDI can be greater than 70 and may be great than 80.

In some aspects, the protein source can be greater than 40% protein and have a moisture content of from about 2% to about 10%, and can have a moisture content between 2% and 7%.

In some aspects, the protein based adhesive of the present process comprises a curative. The curative may also be known as or referred to as a cure additive or crosslinking agent or even as a catalyst. The curative may provide additional properties or manipulate existing properties of the protein component, such as water resistance, solubility, viscosity, shelf-life, elastomeric properties, biological resistance, strength, and the like. Kumar et al., Adhesives and plastics based on soy protein products, Industrial Crops and Products 16 (2002) 155-172, describes various aspects of curing protein adhesives.

Curatives may also be materials that react with some portion of the protein source, enhance the cure of the protein source, co-cure with the protein source, or cure separately but as a network with the protein source. Examples of curatives can be found in U.S. Pat. No. 8.147.968 B2 and include epoxies, isocyanates, sulfur compounds, aldehydes, anhydrides, silanes, azididines, and azetidinium compounds and compounds with all such functional groups. Possible formaldehyde-containing crosslinking agents include formaldehyde, phenol formaldehyde, urea formaldehyde, melamine urea formaldehyde, melamine formaldehyde, phenol resorcinol and combinations thereof.

The curatives of the present process are not limited to the above list and include other curatives that are known in the art. For example polyvinyl acetate latexes and similar compounds can be used. Other curatives include polyamidoamine-epihalohydrin (PAE) type resins.

In other aspects, the curative is a crosslinking agent that has several reactive sites per molecule. The type and amount of curative used in the present process depends on the desired properties. Additionally, the type and amount of curative depends not only on the characteristics of the lignocellulosic material, but may also depend on the protein source used in the adhesive.

In yet other aspects, the curative may or may not contain formaldehyde. In one embodiment, the curative is formaldehyde free or not manufactured using formaldehyde. Although formaldehyde-free curatives are highly desirable in many interior applications, formaldehyde-containing curatives are acceptable for some exterior applications.

In one aspect, the process leads to a composite that emits less than about 0.09 ppm formaldehyde and can be less than about 0.05 ppm using the “large chamber” test method described by the California Air Resource Board (CARE) and based on the test method ASTM E 1333-96(2002). A formaldehyde scavenger may be added to neutralize the formaldehyde that may be emitted by the lignocellulosic material.

In another aspect, the formaldehyde-free curative comprises a PAE resin in amounts ranging from about 0.3% to about 10% of the lignocellulosic component of the composite of a dry weight basis, and can be from about 0.5% to about 5% and can be from about 1% to about 3% and may be from about 1% to about 2.5% and the ratio of PAE to protein can be from about 0.2 to 1, to about lto 1, and can be from 0.3 to 1, to about 0.7 to 1, and the PAE resin contains less than 0.1% of l,3-dichloropropanol.

Amine-epichlorohydrin resins are defined as those prepared through the reaction of epichlorohydrin with amine-functional compounds. Among these are polyamidoamine-epichlorohydrin resins (PAE resins), polyalkylenepolyamine-epichlorohydrin (PAPAE resins) and amine polymer-epichlorohydrin resins (APE resins). The PAE resins include secondary amine-based azetidinium-functional PAE resins, examples include but are not limited to, Kymene™ 55711, Kymene™ 557LX, Kymene™ 913, Kymene™ 920 and ChemVisions™ CA1920, all available from Solenis LLC, Wilmington Del., tertiary amine polyamide-based epoxide-functional resins and tertiary amine polyamidourylene-based epoxide-functional PAE resins, examples include but are not limited to, Kymene™ 450, available from Solenis LLC, Wilmington Del. A suitable crosslinking PAPAE resin is Kymene™ 736, available from Solenis LLC, Wilmington Del. Kymene™ 2064 is an APE resin that is also available from Solenis LLC, Wilmington Del. These are widely used commercial materials. It is also possible to use low molecular weight amine-epichlorohydrin condensates as described in Coscia (U.S. Pat. No. 3,494,775) as formaldehyde-free crosslinkers. It is also feasible to use the low viscosity and high solids resins described in US Patent Publication No. 2011/0190423.

Other additives may be included in the composite and may be incorporated into the adhesive formulation such as extenders, viscosity modifiers, defoamers, diluents, catalysts, tack modifiers, foiinaldehyde scavengers, biocides, pH modifiers, and fillers. Urea may be added for numerous reasons as discussed above in the background section. If urea is added it can be pre-dissolved in water or pre-dissolved into a PAE solution. It can be added with other wet components or added separately. Many particleboard mills spray urea onto the composite furnish to lower formaldehyde emissions from the final board.

In one aspect of the current process, urea is added to the lignocellulosic/curative mixture wherein the level of urea is from 0 to about 5% of the lignocellulosic component of the composites on a dry weight basis, can be from about 1% to about 4%, and may be from about 1% to about 3% dry weight basis. In one embodiment the urea is sprayed onto or mixed with the lignocellulosic separately from the curative and/or the protein source.

In description of the protein source the term “dry” or “powdered” are used interchangeably. These terms do not mean the exclusion of all water because lignocellulosics such as wood, and protein sources such as soy flour, naturally contain water and are usually in a constant state of change of moisture content as they adjust to an equilibrium with the moisture in the air around them. As used in this application, “dry” means the material has no free water that would be released by squeezing and the material is free flowing, on a bulk scale, as a solid. “Dry” also means the solids are moveable as particles and there is no visible water. In a different context, “dry” when referring to the percentage of a formulation means the solids portion of an aqueous material or the weight of the material remaining after drying.

Another aspect the current invention allows greater latitude for the solids concentration of the liquid components added to the composite. The addition of the protein source as a powder, with typically less than 10% moisture content, reduces the amount of solids material that needs to be added in the form of a water solution or dispersion. The moisture contents typically used in the preparation of composites and the limitations and issues associated with moisture content have been known in the composite industry for a long time. Too much moisture during the hot pressing of a composite, such as particleboard, can cause too much steam pressure to build up inside the composite structure such that when the pressure is released the composite blows apart, blisters and/or delaminates. Literature on the preparation of composites, such as particleboard, describes the typical operating window for moisture content. Manufacturers often adjust the moisture content of the lignocellulosic component. A large portion of every particleboard mill is the dryer sections used to control the moisture content of the wood prior to it being treated with adhesive. Often wood is dried to less than 3% moisture content. Even with a low moisture content for the lignocellulosic component there needs to be a limit on the moisture added with the adhesive and other components. The current process provides for more consistency and helps alleviate many of the issues just touched upon.

As an example, consider a wood based composite with wood of 3% moisture content (MC), a protein source as a 33% dispersion, urea as a 33% solution, and a PAE resin at 50% solids. In addition, the dry weight % of the wood is 92%, the dry weight % of the protein is 4%, the dry weight % of the urea is 2%, and the dry weight % of the PAE is 3%. Given these parameters the MC of the final composite prior to heated curing or other drying can be determined as: a) 92 parts wood and thus 2.85 parts water from the wood; b) 4 parts protein source and thus 8 parts water from the protein source; c) 2 parts urea, and thus 4 parts water from the urea; and d) 3 parts PAE curative at 50% solids, and thus 3 parts water from the curative. The total solids material would be 100 parts, and the total water added would be 17.85 parts. This is far more water than can be generally tolerated in manufacturing of a heat cured composite, and the result would be too much pressure built up within the composite structure during curing, which in term would lead to the composite blowing apart upon release of the pressure applied to form the structural shape during curing. Those familiar with manufacturing of composite structures such as particleboard and MDF will understand such a limitation. This amount of water would lead to catastrophic failure of the composites structural shape during curing.

Conversely, if the water needed in the application of the protein source were reduced from 8 parts to 0.21 parts, by adding the protein source in powder form with 5% MC, then the total water content in the above example would be reduced from 17.85 parts by 7.79 parts or a total of 10.06 parts. This is well within the limits for most composite manufacturing to occur without the composite blowing apart on release of the pressure after hot pressing. By decreasing the water level added with the urea or the curing agent, the level of moisture content allowable in the wood could be increased allowing for greater flexibility in board manufacture.

In one embodiment of the current method, the total moisture content of the composite on a basis of the total composite weight prior to pressing can be from about 6 percent to about 13 percent, can be from about 6 percent to about 11 percent and may be from about 6 percent to about 9 percent.

In one embodiment of the method, the total MC in the composite arising from just the lignocellulosic, curing agent, and protein source on a percentage of the total weight of just these materials, in the form in which they are used for the composite before any mixing, is between about 3 percent and 8 percent by wt . . . . That is when the protein source, lignocellulosic material and curative are considered individually, although added separately, contribute a certain level of moisture to the composite structure. This level can be kept low because of the use of a protein source in powder faun. Yet, the current process allows for adequate mixing of the curative and protein source and lignocellulosic despite these materials contributing a low level of moisture. As an example, a composite may have on a relative weight basis 93 parts wood with 3% MC, 5 parts protein source with a 5% MC, and 2 parts curative at 50% MC. The moisture content from just these materials would be (93×0.03+5×0.05+2×0.50)/(93+5+2) or 4.04%. If in the final composite, prior to pressing, these 3 materials made up 95% of the composite, then the MC of the final composite contributed by these materials would be 3.84%. By keeping the moisture content contributed from these materials low, other materials and water may be added to effect characteristics of the final composite and of the process. For example, it can be advantageous to spray water onto the surface of the composite structure prior to curing to provide better heat transfer through steam generation into the center of the composite and it can be advantageous to apply a water based release aid to the surfaces of the composite or it may be advantageous to add a water based urea solution, or water based diluent or water based internal release aid, or a water based latex or water based polymer latex that might enhance properties or a water based catalyst or a water based wax or other compound to enhance water resistance, or a water based fire retardant, or a water based catalyst for enhance curing, or any combination of such materials.

In other aspects of the current process, urea can be added together or separate from the curative, diluents and/or other additives. Adding the urea separately provides the advantages of not having to premix the urea with other materials, it makes use of the urea addition lines already existing in many composite manufacturing mills and it allows separate adjustment of the level of urea which acts as a scavenger for formaldehyde.

In another aspect, the concentration of the urea solution can be greater than about 35% and can be greater than about 50%. Even with the use of a formaldehyde free adhesive the urea can scavenge the formaldehyde given off by a lignocellulosic material.

In another aspect of the current process, the PAE resin has a solids content greater than about 40% and can be greater than about 50%. In one embodiments the curative has a solids content greater than 40%.

In yet another aspect, the curative has a solids content of greater than about 50% and a viscosity less than 2000 cps as measured on a Brookfield LV Viscometer, Model DV-II+ Pro at 12 rpm using a No. 2 spindle. This makes the material easily sprayable and also contributes less moisture to the composite than a curative of lower solids content.

In one embodiment diluents can be added to the composite, such as non-urea diluents that are low volatility, water-soluble or water-dispersible compounds that preferably give low viscosity in water. Non-urea diluents are low volatility, water-soluble or water-dispersible compounds that preferably give low viscosity in water. Non-limiting compounds include diethylene glycol, propylene glycol, 2-methoxyethanol, glycerol and glycerol derivatives, ethylene carbonate, propylene carbonate, methylpyrollidone, low molecular weight polyethylene glycol and derivatives like methoxy polyethylene glycol; sucrose, lactose, sorbitol, maltodextrin, cyclodextrin, carbohydrate, syrups and hydrolyzed low molecular weight polysaccharides or oligosaccharides; inorganic salts such as sodium sulfate, sodium phosphate, and sodium chloride; alum, bentonite, aluminosilicates, and alkali metal aluminosilicates; water soluble organic compounds, such as formamide and acetamide; N-methylpyrrolidinone; surfactants, emulsifiers, and oils, such as vegetable oil, silicon oil, mineral oils and other oils; and mixtures of the above.

The diluent can also be a compound that contains alcohol functionality. The diluent may contain multiple alcohol functionality on the same molecule such as dials and polyols. Some non-urea diluents are glycerol, sucrose, sorbitol, corn syrup, and hydrogenated corn syrup.

In one embodiment of the invention the diluent is a compound that contains alcohol functionality. Preferably the diluent contains multiple alcohol functionality on the same molecule such as dials and polyols.

In some embodiments of the invention the non-urea diluents can be glycerol, sucrose, sorbitol, corn syrup, and hydrogenated corn syrup; and can be molasses

The non-urea diluent can be present in an amount of from about 0.01 to about 75% by weight based on the total wet weight of the adhesive composition. Typically, the non-urea diluent is present in an amount from about 5 to about 60% by weight and more typically from about 10 to about 50% by weight based on the total wet weight of the adhesive composition.

### Tack

Tack, also known as green strength, is that ability of the unset and uncured but formed or shaped composite to hold its shape and remain cohesive from the time the composite is formed or shaped to the time it is set-up or cured or hardened. This is an important property in the manufacture of composites and in the current process, setting-up means the transformation by which the adhesive goes from a liquid to a solid and generally is the point where substantial strength is developed (more than tack) in the formed structure. The adhesive may set-up by different means such as loss of water or by a curing mechanism, such as a chemical reaction. Tack can also be considered the strength of the formed composite prior to curing.

Tack may be measured in different ways. For the purpose of the current process it is measured, and thus defined, by forming a composite structure and testing its integrity. A 3″ by 10″ by ‘/” rectangular structure (sheet) is formed. The structure is made by combining the primary material of a composite (such as wood particles) with an adhesive, by the process of this invention or some other process, and then a certain weight of the uncured composite/adhesive mixture is measured and placed in a 3″×10″ (inner dimension) frame at a uniform thickness and then while still in the frame the uncured material is pressed with 6000 pounds of pressure (200 psi) to form a shaped structure. The frame is removed without disturbing the shaped structure. The shaped structure is made with a metal platen below it and between the platen and the structure is a thin pliable plastic sheet. The platen allows the foitued uncured composite to be moved without influencing the tack results. After pressing, the platen, the plastic, and the structure are moved to a table. The edge of the platen is aligned with the edge of the table. The formed structure, which is riding on the plastic, is then slowly pulled over the edge of the table with the longest length of the structure perpendicular to the edge of the table. The pull over the edge of the table is done at a steady rate of about 1 cm/sec. The structure is moved by pulling the plastic sheet below it over the edge and downward from the top of the table. As each structure extends off the edge of the table it reaches a point at which it cannot support its own weight. The structure extending off the end of the table will break off and fall. The distance a sample extends off the end of the table before breaking off is taken as a measure of tack. The longer a sample extends over the edge, the higher the tack, that is, the more integrity it has. Samples are compared to get a relative effect. Samples are compared to control sample(s) prepared at the same time under the same conditions.

The tack test must be adapted slightly for different types of samples which may vary in ways such as adhesive content, composition, sample thickness, pressure used to form, moisture content, and rate of pull off the table.

One aspect of the current invention is that for a given overall uncured composite moisture content, it leads to enhanced tack versus the wet addition of all of the components.

### Composites

The present invention relates to formed composite structures comprised of a primary material as described above and held together by an adhesive. The composite can also vary in the level of primary materials and adhesive materials as described above. In the current method, the composite is formed into a structure. The composite structures can take many forms from functional shapes, such as bowls, to large sheets such as used to make board products. The structures can be formed from, but are not limited to, loose particles or flakes treated with adhesive or fibers treated with adhesive. The general shape of the composite structure is typically formed after combining the primary material, such as a lignocellulosic material, with the adhesive component(s), but other additives may be applied after the structure is shaped or formed and before extensive curing occurs. In addition to the primary material and adhesives, the composite can contain other additives such as waxes, dyes, catalysts, catalysts for the curing of the adhesive, other fillers, flame retardants, biocides, and other additives known in the composite industry. The wet portion of the adhesives may also contain these materials in either soluble or dispersed form and the additives may be premixed with the adhesive or added at the same time as the adhesive. Powder or dry additives may also be added to the dry protein source. For example, inorganic fillers such as clay or calcium carbonate may be added; or organic fillers such as ground nut shells, or other modifiers such as catalysts for curing, or modifiers such as starch may be added. Such additions may modify properties, curing, tack, cost, or other properties. The composites may also contain diluents, some diluents may alter cure properties, while others may act as plasticizers, and others may be present to increase the solids of the composition, and others may alter rheological properties. The composite and wet or dry portions of the adhesive compositions may also contain a scavenger for formaldehyde. One such example is urea and another is dimethylurea. In some aspects, the diluents and liquid plasticizers of the current process can be added to the composite mixture prior to the addition of the protein source.

The current invention is illustrated by the following examples which describe some embodiments of the invention. However, it should be appreciated that the examples are illustrative only and are not meant to be limiting as to the scope of the invention.

### EXAMPLES

For the examples below Modulus of Rupture (MOR) was measured by cutting 10″×10″ samples into 8 8″×1″ strips. All were ½″ thick. The samples were all cut in the same direction and the same side was kept on top for a 3 point bend test. A mechanical tester from Instron MTS Insight 5 was used for a 3 point bend test and the MOR was taken as the maximum stress at the point of breaking.

Tack was measured by preparing the composite furnish in the same way as for MOR testing, and as described in the examples below. A sample of 90 grams of a lignocellulosic material treated with a curative was placed uniformly in a 3″×10″ mold (a 3″ by 10″ by 1″ frame open on top and bottom) and pressed at room temperature with 200 psi pressure for 5 seconds to press the treated material into ¼″ flat sheet structure. The sample was on a plastic sheet during this process. The sample was then released from the mold and carried on the plastic sheet to a flat surface of a platform for testing. Testing was carried out by pulling the plastic sheet downward at the edge of the platform, a narrow edge of the composite moved off the platform and extended off the edge, suspended by its strength (tack). The samples extension was steadily and slowly increased by pulling on the plastic sheet. The extension of a sample was continued until the part of the composite extending off the platform fell away by its own weight. The length of extension off the edge of the table before falling away was taken as a measurement of tack and report in centimeters. Multiple samples for each condition were tested and averaged to get statistically meaningful results.

### Example 1

### Wet Process vs. Dry Process

Example one illustrates that the preparation of a wood particleboard is simplified by use of the process of the current invention (dry process) versus the traditional process of mixing soy flour and water and adhesive components. At the same time the dry process leads to enhanced strength of the composite. Strength for this example refers to the Modulus of Rupture of the particleboard as measured with a three point bend test.

### Example 1a

A traditional composite process was used as follows: 293 g water was mixed with 76.5 g of glycerol (98% solids), 0.6 g of a commercial defoamer, Advantage® 357, (Solenis LLC, Wilmington, Del.) and 1.5 g sodium meta bisulfite. To this mixture, with mixing, was slowly added 160 g soy flour, Prolia® 200/90 (6.2% moisture content) (Cargill, Minneapolis, Minn.) until the soy flour was thoroughly mixed in. The pH of the complete mixture was lowered to below 3.8 by the addition of 11.7 g 50% sulfuric acid to eliminate any urease in the soy flour. The mixture was held at a low pH for 4 hours, and then transferred through a course paint filter or strainer, such as a Gerson 2K Paper Paint Strainer #252 or Astro Pneumatic AST4583F Nylon Mesh Paint Strainer, to another jar. Then 75 g of urea was added to the mixture along with 0.06 g of a biocide. The final solids content was about 50%. From this mixture, 150.6 g was combined with 40.65 g of a 55% solids low viscosity PAE resin, and 2.37 g water. The PAE resin was prepared according to U.S. patent application Ser. No. 13/020,069 filed Feb. 3, 2011, published as US 2011/0190423. This combination of soy flour and PAE was used within 20 minutes of mixing.

Next, 636 g of wood furnish with 8% moisture content, in the form of particles, {such as used for making the core of particleboard) was placed in a Bosch 800 Watt Universal Plus Mixer, model MUM6N11 fitted with the manufacturers cookie dough paddles. The wood was stirred at the mixer's lowest speed and while being stirred the wood was sprayed from above with 92.87 g of the above mixture of soy flour, glycerol, urea, and PAE resin. The spraying was done over about a 2 minute time period and was followed by 10 seconds of mixing in the blender. The mixture was then allowed to sit in a covered container for 10 minutes and was then prepared into a particleboard sample by forming, cold pressing, and finally hot pressing.

### Example 1b

The process according to the current method was used as follows: 636 g of wood furnish at 8% moisture content, the same type as used in Example 1a, was sprayed with 73.6 g of the following mixture: 46.16 g PAE resin {the same as used above at 55% solids), 21.25 g urea, 21.68 g glycerol (at 98% solids), and 84.55 g water. Spraying occurred as the wood was mixing. After spraying the wood the mixture was stirred for 10 seconds. The treated wood was transferred to a separate container and onto the wood was added all at once 19.23 g Prolia 200/90 soy flour having a 6.2% moisture content. The treated wood and soy flour were then mixed by hand for about 20 seconds. With the “dry” process there was no need to acid treat the soy, or add sodium meta bisulfite to lower the viscosity, and no defoamer was added.

### Example 1c

Example 1b was repeated except instead of the treated furnish being mixed with soy flour, the wood furnish and soy flour were mixed for 10 seconds prior to the wood being sprayed by the liquid components.

In each of the Examples 1a, 1b and 1c, the water level was adjusted such that the composites treated furnishes (wood plus soy flour plus curative plus additives) for each sample had the same overall moisture content. This was done to obtain results uninfluenced by water content, which can affect sample cure and density. The PAE mixture was sprayed onto the wood/soy mixture in the same manner as the wet soy dispersion/PAE mixture of Example 1a, as just described. The lower viscosity of the liquid materials that did not contain soy flour made them easier to spray. The spray time and post spray mix times were the same as for the wet process.

Each treated composite furnish for examples 1a, 1b, and 1c was handled and treated the same in the preparation of a particleboard. 636 g of each treated composite furnish was placed in a 10 inch by 10 inch frame, leveled, and cold pressed. The frame was then removed and the structure was hot pressed to a ½ inch thickness using shims. The press conditions were 160° C., for 210 seconds. Each sample after pressing was cooled on a rack to room temperature and after 5 minutes at room temperature was sealed in a bag to maintain constant moisture until being cut and tested.

The Modulus of Rupture (MOR) of Examples 1a, 1b and 1c was determined with a three point bend test. Eight samples of each example were tested and the strength values averaged. Each sample was 8″ long, 1″wide and 0.5″ thick. The preparation and testing on all three examples were repeated and the values for the experiments were averaged.

Boards that were made using the method outlined in Example 1a, had an MOR of 1582 psi. The boards of Example 1b, had an MOR of 2148 psi and those of Example 1c, had an MOR of 2098. These Examples demonstrate that the “dry” process gives a surprising increase in strength from identical formulations. Furthermore, as can be seen in Table 1, a separate post addition of the soy flour after first mixing the lignocellulosic and the curative gave enhanced strength above premixing the soy flour with the wood.

TABLE 1
Modulus of Rupture and Tack Results

| Process | Average MOR (psi) | Tack (cm overhang) |
|---|---|---|
| Wet | 1582 | 3.10 |
| Dry with post added soy flour | 2148 | 5.42 |
| Dry with pre added soy flour | 2098 | 4.71 |

For each of Examples 1a, 1b and 1c, the tack of the treated furnish was evaluated using the method described above. The same sample preparation conditions as used in Examples 1a, 1b and 1c, were used in the following Example. Several 3″×10″×¼ samples of each process were prepared, cold pressed at 200 psi for 5 seconds while still contained in a mold, and then tested for tack. The results are listed in the table above. The dry-processes provided greater tack than the wet process, and post addition of the soy flour led to more tack than pre-addition of the soy flour.

### Example 2

### Variation of Moisture Content

Particleboard may have better strength properties under certain conditions. Example 2, shows that, in keeping with the current method, higher moisture content, within bounds of normal particleboard manufacturing, will lead to better strength properties.

The method of post addition of soy flour, as in Example 1b, was used in this Example. As with Examples 1a, 1b and 1c, the soy flour was Prolia® 200/90 from Cargill.

The only variable for this example was the amount of water added into the materials that were sprayed. By varying the water level of the additives, the moisture content of the furnish prior to hot-pressing was varied. The moisture contents of the formed composite samples were 8.8, 10.5, and 12.5 parts per hundred parts dry wood. The same adhesive formulation and level, 7.5 parts per 100 parts dry wood, was used in each case. The samples were pressed at 160° C. for 210 seconds. For each condition, a sample was also made with the soy flour added to the lignocellulosic material prior to the spraying of the liquids.

The following table lists the strengths obtained. The results show that a higher moisture content, within the parameter of the experiment, led to stronger boards. As is known in the industry, the board strength is highly dependent on board density, and higher moisture contents led to more density. However, density was not the only factor and clearly a high moisture content even at the same density led to greater strength. As can be seen in Table 2, the process with post addition of soy flour was more tolerant of lower moisture contents than pre-addition of the soy flour.

TABLE 2

| Moisture | Post soy flour | Board | Pre soy flour | Board |
|---|---|---|---|---|
| Content | addition | Density | addition | Density |
| 8.8 | 1563 | 44.6 | 1404 | 44.0 |
| 10.5 | 1846 | 44.9 | 1661 | 44.8 |
| 12.5 | 1799 | 44.9 | 1837 | 44.9 |

### Example 3

### Various Protein Source

Various protein sources may be used in the protein portion of the adhesive used in the process of the current invention. Likewise various lignocellulosic materials may be used.

The same post protein source addition process used for Example 2 was used to make the following samples. The board adhesive level was 8 pph of the dry-wood. The moisture content before hot-pressing was 12.5%. The same press conditions were used as in previous Examples.

The same formulation as in Example 2 was used, except the soy flour was replaced by other protein sources. Table 3, lists the various protein sources and the resulting MOR values obtained. As with Example 1, the experiment was repeated and the values averaged. With all of the protein sources the process successfully produced a particleboard sample.

TABLE 3
Modulus of Rupture Results

| protein source | MOR |
|---|---|
| Lupin Flour | 1551 |
| Peanut Flour | 1659 |
| Whey Protein | 1674 |

### Example 4

### Variation of Soy Flour

Using the formulation and post soy flour addition process of example 2, with 8pph adhesive and 10.5% moisture content, this example presents the effect of changing soy flour characteristics. Four different soy flours were used to prepare particleboard samples using identical conditions. The soy flour types were 200/90, 200170, 200/20, and 100/90. The first number in each type name describes the fineness of the grind by mesh size and the second number describes the soy flour PDT. The MOR values obtained with each type are shown in Table 4. The best results were obtained with 90 PDI soy flour materials.

TABLE 4
Modulus of Rupture of Differing soy flour PDI's

| Soy Flour Type | MOR |
|---|---|
| 200/90 | 1814 |
| 200/70 | 1716 |
| 200/20 | 1730 |
| 100/90 | 1795 |

### Example 5

The premix of Example 1a, with soy flour, glycerol, and urea was repeated. An identical premix was prepared except dimethylurea was used in place of urea. Dimethylurea does not break down to ammonia in the presence of urease. The rest of Example 1a was followed using this new premix and a repeat of the 1a premix, the only differences being that in this example the adhesive level used was 8% and the moisture content of the treated wood was 13.3% prior to pressing.

The formulation of Example 1b was followed. In addition, an identical formulation was prepared with dimethylurea in place of urea. The rest of Example 1b was followed, with the adhesive level being 8% and the moisture content of the treated wood being 13.3% prior to pressing. Thus four variations were compared using the same formulations—Sample 1., a wet process with urea, Sample 2., a wet-process with dimethylurea, Sample 3., the dry-process of this invention with urea, and Sample 4., the dry-process of this invention with dimethylurea. The final composite samples, which were made in the same manner as the samples in Examples 1a, 1b and 1c, were evaluated for the strength by a measure of MOR. All four samples were repeated and the results averaged with those of the first samples. The averaged results are in Table 5.

TABLE 5
Modulus of Rupture with Urea Additive

| | |
|---|---|
| Sample | MOR |

| | | |
|---|---|---|
| 1. | wet-process with urea | 1595 |
| 2. | wet-process with dimethyl urea | 1537 |
| 3. | dry-process with urea | 1925 |
| 4. | dry-process with dimethyl urea | 1792 |

The use of dimethyl urea (Samples 2. and 4.) versus urea (Samples 1. and 3.) made a small difference in the wet-process where the urease was inactivated by an acid treatment of the soy flour. Both wet-process Samples 1 and 2, had inferior strength compared with either of the dry-process Samples 3 and 4, thus showing that both the process itself and the ability to generate ammonia are important for improving the strength of the boards. Of the two dry-process samples, better strength was seen for Sample 3, containing urea, where ammonia was generated giving a pH increase.

### Example 6

The formulation of Example 5, Sample 3, with the dry process and urea was prepared. The curative was mixed with urea and sprayed onto the wood particles at the same time the soy flour was added as would be typical on a continuous particle board line and the additions occurred in an industrial blender. Blender paddles rotating quickly mixed and advanced the wood through the blender in a few seconds. A continuous supply of untreated wood entered the blender and a continuous supply of treated wood exited the blender. After the process was running for several hours it was stopped. The inside of the blender was examined and there was observed a buildup of material on the walls near the point of spray of the liquids. The buildup contained a high level of soy flour and other components of the composite. The buildup of a deposit did not occur when soy flour was not added to the blender.

### Example 7

Wood chips of a mixture of spruce, pine, and fur were processed into wood fibers suitable for MDF manufacturing by a pressurized steam/refining process. The fibers were injected into an MDF manufacturing blow line. At the start of the blow line part A of a soy flour based adhesive was sprayed from a water solution into the blow line and thus onto the fibers. After the fibers were mostly dry by passing through the blow line (to a moisture content of 6.6%), soy flour (part B of the adhesive) was added and mixed with the treated fibers. The treated fibers were then formed into a mat, and pressed at 180C for 3 minutes to form a panel of MDF.

Part A consisted of a mixture of (A) 72.0 parts at 55% solids of a high solids, low viscosity PAE resin from Solenis (Soyad™ CL4180); (B) 23.6 parts glycerol at 98% solids; (C) 37.3 parts urea at 100% solids, (D) 21.4 parts at 58% solids of a wax emulsion typically used in wood composite manufacturing and (E) enough water to give desired solids. The total solids of the Part A mixture was 52% and the amount added to the fiber was 8.4 parts on a dry basis to 100 parts dry fiber. Part B of the mixture was a 90 PDI soy flour ground to pass 90% through a 200 mesh screen. The amount of soy flour added to the fiber was 5.6 parts on a dry basis to 100 parts dry fiber.

The board formed by the process of this example had a thickness of 13 mm and a density of 42 pounds per cubic foot. It gave a Modulus of Rupture (a 3 point bend test strength) of 2654 psi. No issues such as blows were encountered in the hot presses. Therefore, the process was successfully used to form a useable MDF sample of good strength.

Each reference cited in the present application above, including books, patents, published applications, journal articles and other publications, is incorporated herein by reference in its entirety.

We claim:

1. A method of preparing a lignocellulosic based composite comprising:
a. combining at least one lignocellulosic material with one or more curatives forming a mixture, wherein at least one of the one or more curatives is dissolved or suspended in water;
b. adding at least one powdered protein source to the mixture;
c. forming the composite;
d. curing the formed composite.

2. The method of claim 1, wherein the curative and lignocellulosic material are combined in a blender and the powdered protein source is added to the mixture after the mixture exits the blender.

3. The method of claim 1, wherein the dissolved or dispersed curative and the lignocellulosic material are combined in a blowline and the powdered protein source is added after the mixture exits the blowline.

4. The method of claim 1, where the protein source has a protein content greater than 40% by wt.

5. The method of claim 6, wherein the protein content is greater than 70% by weight.

6. The method of claim 1, where the protein source has a PDI greater than 80.

7. The method of claim 1, wherein the mixture further comprises urea.

8. The method of claim 1, wherein the level of urea on a dry basis is from about 1 part to about 4 parts per 100 parts lignocellulosic material.

9. The method of claim 1, wherein the protein source contains urease.

10. The method of claim 1, wherein the powdered protein source has an average particle size of less than about 50 microns.

11. The method according to claim 10, wherein the powdered protein source has an average particle size of less than about 25 microns.

12. The method of claim 1, wherein the protein source is from about 1.6 to about 10 parts per hundred of the lignocellulosic component.

13. The method of claim 12, wherein the protein source is from about 4 parts per hundred to about 6 parts per hundred of the lignocellulosic component.

14. The method of claim 1, wherein the curative is a polyamidoamine-epichlorohydrin resin having azetidinium functional groups capable of reaction with protein.

15. The method of claim 1, wherein the level of curative on a dry basis is from about 30 parts to about 100 parts per 100 parts protein source.

16. The method of claim 1, wherein at least one additive is added to the mixture prior to addition of the protein source.

17. The method of claim 1, where the moisture level of lignocellulosic material can be higher than 5% prior to combining with the curative.

18. The method of claim 1, where the total moisture content of the lignocellulosic material, curative and protein source in the state in which they are combined with other components is less than about 8%.

19. The method of claim 1, wherein the formed and/or cured composite is at least one layer of a multilayer composite.

20. The method of claim 1, wherein the cured composite is selected from the group consisting of particleboard and medium density fiber board (MDF).
